SEQUENCE LISTING (13 sequences)

SEQ ID NO: 1 - RNA, 31 nt, Artificial
  an artificially synthesized target sequence
uauuuuuuuagguagcggguaguaguuguuu

SEQ ID NO: 2 - RNA, 31 nt, Artificial
  an artificially synthesized capture probe sequence
aaacaacuacuacccgcuaccuaaaaaaaua

SEQ ID NO: 3 - RNA, 31 nt, Artificial; identical to SEQ ID NO: 2
  an artificially synthesized capture probe sequence

SEQ ID NO: 4 - RNA, 31 nt, Artificial
  an artificially synthesized target sequence
uauuuuuuuagguaguggguaguaguuguuu

SEQ ID NO: 5 - RNA, 31 nt, Artificial
  an artificially synthesized capture probe sequence
aaacaacuacuacccacuaccuaaaaaaaua

SEQ ID NO: 6 - RNA, 31 nt, Artificial; identical to SEQ ID NO: 5
  an artificially synthesized capture probe sequence

SEQ ID NO: 7 - DNA, 375 bp, Artificial
  an artificially synthesized sequence
atcacctcagcagaggcacacaagcccggttccggcatctctgctcctattggctggatatttcgtattccccgagctcctaaaaacgaaccaataggaagagcggacagcgatctctaacgcgcaagcgcatatccttctaggtagcgggcagtagccgcttcagggagggacgaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatg

SEQ ID NO: 8 - DNA, 21 bp, Artificial
  an artificially synthesized primer sequence
atcacctcagcagaggcacac

SEQ ID NO: 9 - DNA, 21 bp, Artificial
  an artificially synthesized primer sequence
tttggcgccagaagagccaag

SEQ ID NO: 10 - DNA, 41 bp, Artificial
  an artificially synthesized primer sequence
taatacgactcactatagggattattttagtagaggtatat

SEQ ID NO: 11 - DNA, 21 bp, Artificial
  an artificially synthesized primer sequence
tttggtgttagaagagttaag

SEQ ID NO: 12 - DNA, 41 bp, Artificial; identical to SEQ ID NO: 10
  an artificially synthesized primer sequence

SEQ ID NO: 13 - DNA, 21 bp, Artificial
  an artificially synthesized primer sequence
tttggtgttagaagagttaag

(19) United States
(12) Patent Application Publication
(10) Pub. No.: US 2006/0110741 A1
(43) Pub. Date: May 25, 2006

(54) Nucleic acid methylation detection process using an internal reference sample

(76) Inventors: Ryoichi Asai, Tokyo (JP); Katsutoshi Takahashi, Tokyo (JP); Ali Arjomand, Tokyo (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/540,897

(22) PCT Filed: Dec. 25, 2003
(86) PCT No.: PCT/JP03/16716

§ 371 Date: Nov. 28, 2005

(30) Foreign Application Priority Data
Dec. 25, 2002 (JP) 2002-375102

(51) Int. Cl. C12Q 1/68; C12P 19/34

(52) U.S. Cl. 435/6; 435/91.2

(57) ABSTRACT

There is disclosed a process for detection of DNA methylation at CpG sites using nucleic acid arrays and preferably microarrays. Specifically, there is disclosed a process for directly generating a reference sample from the sample to be tested and detecting methylation at large numbers of CpG island sites simultaneously. More specifically, the inventive process comprises dividing a DNA sample into two samples (a first sample and a second sample), amplifying the first DNA sample by a nucleic acid amplification process such that any methylcytosine residues are amplified as unmethylated cytosine residues, treating the amplified first sample and the (unamplified) second sample with bisulfite to convert unmethylated cytosine residues in both samples to deoxyuracil residues, labeling the bisulfite-converted second sample with a second fluorescent marker and the bisulfite-converted first sample with a first fluorescent marker, wherein the first and second fluorescent markers have non-overlapping fluorescent excitation and emission spectra; and hybridizing the first sample and the second sample onto a microarray device having a plurality of oligonucleotide capture probes designed to hybridize to CpG island sites of the DNA sample as converted and non-converted by bisulfite.

### TECHNICAL FIELD

The present invention provides a process for detection of DNA methylation at CpG sites using nucleic acid arrays and preferably microarrays. Specifically, the present invention provides a process for directly generating a reference sample from the sample to be tested and detecting methylation at large numbers of CpG island sites simultaneously. Specifically, the inventive process comprises dividing a DNA sample into two samples (a first sample and a second sample), amplifying the first DNA sample by a nucleic acid amplification process such that any methylcytosine residues are amplified as unmethylated cytosine residues, treating the amplified first sample and the (unamplified) second sample with bisulfite to convert unmethylated cytosine residues in both samples to deoxyuracil residues, labeling the bisulfite-converted second sample with a second fluorescent marker and the bisulfite-converted first sample with a first fluorescent marker, wherein the first and second fluorescent markers have non-overlapping fluorescent excitation and emission spectra; and hybridizing the first sample and the second sample onto a microarray device having a plurality of oligonucleotide capture probes designed to hybridize to CpG island sites of the DNA sample as converted and non-converted by bisulfite.

### BACKGROUND ART

Methylation Assay Processes

Methylation of cytosines (C) in the 5′ position of the pyrimidine ring has been shown to be an important epigenetic determinant if a cell or tissue sample is cancerous. In animals, methylcytosine is mainly found in cytosine-guanine (CpG) dinucleotides, whereas in plants it is most often found in cytosine-any base-guanine (CpNpG) trinucleotide sequences.

Methylation of C residues in genomic DNA plays a key role in regulation of gene expression (Wolffe et al., Proc. Natl. Acad. Sci. USA 96:5894-5896, 1999) because the presence of 5-methylcytosine in the promoter of specific genes alters the binding of transcriptional factors and other promoters to DNA (Costello and Plass, J. Med. Genet. 38:285-503, 2001). Further, 5-methylcytosine in the promoter of specific genes also attracts methyl-DNA binding proteins and histone deacetylases that modify chromatin structure around the gene transcription site. Both effects result in blocking transcription and cause gene silencing (Bird, Nature 321:209-213, 1986).

Generally, levels of methylcystine occurrence in genomic DNA have been measured using two different general processes, including processes employing high-performance separation techniques or by enzymatic/chemical means. In order to perfect large scale screening techniques, the enzymatic/chemical means are preferred because they do not require expensive and complex analytical equipment. However, the enzymatic/chemical techniques have not been as sensitive as high-performance separation techniques and the resolution is often restricted to endonuclease cleavage sites.

Two alternative approaches have been tried for DNA methylation detection, bisulfite methods and non-bisulfate methods. Non-bisulfate methods use methylation-sensitive restriction endonucleases combined with Southern blot analysis or PCR detection, but often results are limited to cleavage sites. Bisulfite modification of DNA allows for quantitative determination of methylation status of an allele and requires PCR amplification of bisulfate-modified DNA. Differences in methylcytosine patterns are displayed by methylation-dependent primer designs (i.e., methylation-specific PCR) in conjunction with methylation-sensitive restriction endonucleases, genomic sequencing or other approaches.

Bisulfite treatment of DNA converts unmethylated cytosine to uracil, while methylated cytosine does not react (Furuichi et al., Biochem. Biophys. Res. Commun. 41:1185-1191, 1970). Bisulfate modification of genomic DNA requires prior DNA denaturation because only methylcytosines that are located in single strands are susceptible to attack (Shapiro et al., J. Am. Chem. Soc. 96:206-212, 1974). However, there are problems associated with bisulfite treatment, including, for example, only partial denaturation (Rein et al., J. Biol. Chem. 272:10021-10029, 1997), renaturation problems in high salt concentrations, and incomplete desulfonation after bisulfate treatment (Thomassin et al., Methods 19:465-475, 1999). Moreover, the total conversion of cytosines to uracils is critical to the analysis, so temperature, time and pH conditions are critical without destroying the integrity of the DNA material.

In bisulfite modification methylation detection processes, the most straightforward way of measuring methylation at CpG islands is by sequencing. However, sequencing techniques are also the most difficult (time consuming and expensive) and do not allow for multiplexing of large numbers of scattered CpG island sites in genomic DNA samples. In general, after denaturation and bisulfite modification of a genomic DNA sample, the resulting dsDNA is obtained by primer extension and the fragment of interest is amplified by PCR techniques (Clark et al., Nucl. Acids Res. 22:2990-2997, 1994). Standard DNA sequencing of the PCR products then detects Methylcytosine. Alternatively, one could clone the PCR products into plasmid vectors followed by sequencing of individual clones for a slowed method but one that could also provide methylation maps of single DNA molecules. In another variation, direct localization of methylcytosines in the product of bisulfite treatment instead of the PCR product can be done using only three deoxynucleotides (dATP, dCTP and dTTP) but lacking dGTP that produces an elongation stop at methylcytosine points (Radlinska and Skowronek, Acta Microbiol. Pol. 47:327-334, 1998).

Another process in the bisulfite class is methylation-specific PCR (Esteller et al., Cancer Res. 61:3225-3229, 2001; and Herman et al., Proc. Natl. Acad. Sci. USA 93:9821-9826, 1996), also called MSP. In normal (non-cancerous) cells, cytosines in CpG islands are usually unmethylated, but they become methylated in the promoter sequences of genes associated with certain abnormal cellular processes, such as cancer (Esteller et al., Cancer Res. 59:793-797, 1999; Esteller et al., Cancer Res. 61:3225-3229, 2001; and Esteller et al., Hum. Mol. Genet. 10:3001-3007, 2001). Bisulfite-converted DNA strands are no longer complementary, so primer design in MSP is customized for each chain and methylation patterns of all sequences determined in separate reactions. MSP uses a difficult PCR process and critical primer designs using a narrow range of strand annealing temperatures, the PCT product is between 80 and 175 base pairs, each primer should contain at least two CpG pairs, the sense pair should contain a CpG pair at the 3′ end and primers contain non-CpG cytosines. The MSP technique requires PCR and if the PCR goes for too many cycles of amplification without ensuring that the reaction is in the lineal response range with respect to template concentration, then large over-estimations of the extent of methylation can be obtained if the sequence is amplifiable with both the methylation-specific primers and the primers for unmethylated sequences.

The MSP method was improved by combining methylation-specific PCR with in situ hybridization (Nuovo et al., Proc. Natl. Acad. Sci. USA 96:12754-12759, 1999) to allow for the methylation status of specific DNA sequences to be visualized in individual cells, for monitoring complex tissue samples having both tumor and normal cells. Another method combines MSP with denaturing HPLC to allow for small cell mosaics of structurally normal or abnormal chromosomes to be detected (Baumer et al., Hum. Mutat. 17:423-430, 2001). Specifically, following PCR amplification, the two alleles can be resolved from the two populations of PCR products by denaturing HPLC because they differ at several positions within the amplified sequence.

Another quantification approach has been called MethyLight and uses fluorescent-based, real-time PCR (U.S. Pat. No. 6,331,393 the disclosure of which is incorporated by reference herein; and Eads et al., Nucleic Acids Res. 28:E32, 2000). The DNA is modified by the bisulfite treatment and amplified by fluorescence-based, real-time quantitative PCR using locus-specific PCR primers that flank an oligonucleotide probe with a 5′ fluorescence reporter dye and a 3′ quencher dye. The reporter is enzymatically released during the reaction, and fluorescence, which is proportional to the amount of PCR product and thus to the degree of methylation, can be sequentially detected in an automated nucleotide sequencer device. While fluorescence increases the sensitivity of this process, the process is difficult, requires expensive instrumentation and consumables and cannot be multiplexed to detected hundreds or thousands of CpG island sites simultaneously.

Another approach has been to combine methyl-sensitive endonucleases with PCR amplification with subsequent hybridization to oligonucleotide microarrays (Huang et al., Hum. Mol. Genetics, 8:459-70, 1999). In this case, methylation state was determined by digestion of unmethylated DNA using methylation sensitive restriction enzyme. Unmethylated DNA was enzymatically digested into fragments and did not generate amplicons after PCR whereas methylated DNA was protected from digestion and did generate amplicons after PCR. The presence or absence of amplicons was detected on oligonucleotide microarrays using fluorescent tags. Samples from normal tissues were used as a control with the supposition that these non-cancerous samples contained predominantly unmethylated cytosine residues. This procedure requires DNA from non-cancerous tissue to be available for use as an external control. Additionally, the exact methylation state of the external control needs to be ascertained before it can be confidently used to interpret results from a dual-hybridization assay.

Another approach has been to perform a dual-hybridization assay using a test sample and an external reference sample known to be unmethylated in the analyzed region (Balog et al., Anal Biochem. 309: 301-310, 2002). In this case, a 190-bp DNA duplex was synthesized and used as an external reference sample, or DNA was obtained from a sample known to be unmethylated. The two samples were labeled with different fluorescent dyes, mixed and hybridized to an array containing 21mer oligonucleotides. The external reference sample generated signal in a reference fluorescent channel on capture probes hybridizing to a thymidine residue. The presence of signal on a capture molecule probing for the presence of C within the test sample indicated methylation of that C residue.

Therefore, there are a variety of methylation detection processes that have advantages and disadvantages, but none have the ability to determine the methylation state of a large number of CpG islands without the presence of an external reference sample. Therefore, there is a need in the art to incorporate processes that do not require an external reference sample yet are able to multiplex DNA methylation assays to simultaneously determine methylation patterns.

DNA Microarrays

In the world of microarrays or biochips, biological molecules (e.g., oligonucleotides, polypeptides, oligopeptides and the like) are placed onto surfaces at defined locations for potential binding with target samples of nucleotides or receptors or other molecules. Microarrays are miniaturized arrays of biomolecules available or being developed on a variety of platforms. Much of the initial focus for these microarrays have been in genomics with an emphasis on cellular gene expression, single nucleotide polymorphisms (SNPs) and genomic DNA detection/validation, functional genomics and proteomics (Wilgenbus and Lichter, J. Mol. Med. 77:761, 1999; Ashfari et al., Cancer Res. 59:4759, 1999; Kurian et al., J. Pathol. 187:267, 1999; Hacia, Nature Genetics 21 suppl.:42, 1999; Hacia et al., Mol. Psychiatry 3:483, 1998; and Johnson, Curr. Biol. 26:R171, 1998).

There are, in general, three categories of microarrays (also “DNA Arrays” and “Gene Chips” but this descriptive name has been attempted to be a trademark) having oligonucleotide content. Most often, the oligonucleotide microarrays have a solid surface, usually silicon-based and most often a glass microscopic slide. Oligonucleotide microarrays are often made by different techniques, including (1) “spotting” by depositing single nucleotides for in situ synthesis or completed oligonucleotides by physical means (ink jet printing and the like), (2) photolithographic techniques for in situ oligonucleotide synthesis (see, for example, Fodor U.S. Pat. No. 5,445,934 and the additional patents that claim priority from this priority document, (3) electrochemical in situ synthesis based upon pH based removal of blocking chemical functional groups (see, for example, Montgomery U.S. Pat. No. 6,093,302 the disclosure of which is incorporated by reference herein and Southern U.S. Pat. No. 5,667,667), and (4) electric field attraction/repulsion of fully-formed oligonucleotides (see, for example, Hollis et al., U.S. Pat. No. 5,653,939 and its duplicate Heller U.S. Pat. No. 5,929,208). Only the first three basic techniques can form oligonucleotides in situ, which are, building each oligonucleotide, nucleotide-by-nucleotide, on the microarray surface without placing or attracting fully formed oligonucleotides.

The electrochemistry platform (Montgomery U.S. Pat. No. 6,093,302, the disclosure of which is incorporated by reference herein) provides a microarray based upon a semiconductor chip platform having a plurality of microelectrodes. This chip design uses Complimentary Metal Oxide Semiconductor (CMOS) technology to create high-density arrays of microelectrodes with parallel addressing for selecting and controlling individual microelectrodes within the array. The electrodes turned on with current flow generate electrochemical reagents (particularly acidic protons) to alter the pH in a small, defined “virtual flask” region or volume adjacent to the electrode. The microarray is coated with a porous matrix for a reaction layer material. Thickness and porosity of the material is carefully controlled and biomolecules are synthesized within volumes of the porous matrix whose pH has been altered through controlled diffusion of protons generated electrochemically and whose diffusion is limited by diffusion coefficients and the buffering capacities of solutions.

The microarrays that are made with oligonucleotide capture probes are generally spotted onto glass slides. However, the glass slides are not well suited for creating a reaction chamber with the capture probes that form the spots as the hybridization reaction of target nucleic acids with the capture probes is long and involves controlled conditions. Therefore, there is a need in the art to create better reaction chambers that allow for control of hybridization conditions including stringency conditions (e.g., temperature, gas pressures, chemical environment and pH).

### DISCLOSURE OF THE INVENTION

In view of the many processes that have advantages and drawbacks for quantitative methylation determination, there is a need in the art for being able to multiplex many different sites or CpG islands for methylation analysis simultaneously and in parallel, preferably using existing DNA microarray technology. The present invention was made to develop a methylation process adapted to DNA microarrays to take advantage of the multiplex capabilities of DNA microarrays for methylation analysis.

The present invention provides a process for detecting methylation at large numbers of CpG island sites simultaneously using a reference sample obtained from the sample to be tested, comprising:

(a) providing a sample of DNA for analysis;

(b) dividing the DNA sample a first DNA sample and a second DNA sample, whereby the first sample will become a test sample and the second sample will become an internal reference sample;

(c) amplifying the second DNA sample by a nucleic acid amplification process such that methylcytosine residues are amplified as unmethylated cytosine residues;

(d) bisulfite converting the amplified first DNA sample and the second DNA sample to convert unmethylated cytosine residues to deoxyuracil residues in both samples;

(e) amplifying the converted first DNA sample and the converted second DNA sample;

(f) labeling the bisulfite-converted second DNA sample with a second fluorescent marker and the bisulfite-converted first DNA sample with a first fluorescent marker, wherein the first and second fluorescent markers have non-overlapping fluorescent excitation and emission spectra; and

(g) hybridizing the first DNA sample and the second DNA sample onto a microarray device having a plurality of oligonucleotide capture probes designed to hybridize to CpG island sites of the DNA sample as converted and non-converted by bisulfite.

Preferably, the amplification technique employed is PCR (polymerase chain reaction). Preferably, the hybridization conditions are high stringency. Preferably, the non-overlapping fluorescent labels are Cy3, (1,1′-bis(ε-carboxypentyl)-1′ethyl-3,3,3′,3′-tetramethylindocarbocyanine-5,5′-disulfonate potassium salt di-N-hydroxysuccinimide ester) and Cy5 (1,1′-bis(ε-carboxypentyl)-1′ethyl-3,3,3′,3′-tetramethylindodicarbocyanine-5,5′-disulfonate potassium salt di-N-hydroxysuccinimide ester); or the non-overlapping fluorescent labels are Alexa Fluor 594 and Alexa Flour 546; or the non-overlapping fluorescent labels are Fluorescene and Texas Red.

The present invention also provides a microarray device for using the process of above-mentioned invention, having a plurality of oligonucleotide capture probes designed to hybridize to CpG island sites of the DNA sample as converted and non-converted by bisulfite, and a kit for the process of above-mentioned invention. Preferably, the kit comprises the microarray device, bisulfite converting reagents and DNA labeling reagents.

Specifically, the present invention provides:

(1) A process that simultaneously detects methylation at multiple CpG island sites using a reference sample obtained from a sample to be tested, wherein the process is a nucleic acid methylation detection process that uses an internal reference sample and comprises the steps of:

using a DNA sample for analysis, that is divided into a first DNA sample to be tested and a second DNA sample to be the internal reference, to amplify the second DNA sample such that methylcytosine residues are amplified as unmethylated cytosine residues;

converting the unmethylated cytosine residues to deoxyuracil residues in both the first DNA sample and the second DNA sample;

using a first fluorescent marker and a second fluorescent marker having non-overlapping fluorescent excitation and fluorescent emission spectra to label the first DNA sample with the first fluorescent marker and to label the second DNA sample with the second fluorescent marker; and

hybridizing the first DNA sample and the second DNA sample onto a microarray device having a plurality of oligonucleotide capture probes designed to hybridize to CpG island sites of the DNA sample as converted and non-converted forms;

(2) A process that simultaneously detects methylation at a large number of CpG island sites using a reference sample obtained from a sample to be tested, comprising:

(a) providing a DNA sample for analysis;

(b) dividing the DNA sample into a first DNA sample and a second DNA sample, whereby the first sample will become a test sample and the second sample will become an internal reference sample;

(c) amplifying the second DNA sample by a nucleic acid amplification process such that methylcytosine residues are amplified as unmethylated cytosine residues;

(d) bisulfite conversion of unmethylated cytosine residues into deoxyuracil residues in both the amplified first DNA sample and the second DNA sample;

(e) amplifying the converted first DNA sample and the converted second DNA sample;

(f) labeling the bisulfite-converted second DNA sample with a second fluorescent marker and the bisulfite-converted first DNA sample with a first fluorescent marker, wherein the first and second fluorescent markers have non-overlapping fluorescent excitation and emission spectra; and

(g) hybridizing the first DNA sample and the second DNA sample onto a microarray device having a plurality of oligonucleotide capture probes designed to hybridize to CpG island sites of the DNA sample as converted and non-converted by bisulfite;

(3) The process of (1) or (2), wherein the amplification technique employed is PCR (polymerase chain reaction);

(4) The process of any one of (1) to (3), wherein the hybridization conditions are highly stringent conditions;

(5) The process of any one of (1) to (4), wherein the non-overlapping fluorescent labels are Cy3, (1,1′-bis (ε-carboxypentyl)-1′ethyl-3,3,3′,3′-tetramethylindocarbocyanine-5,5′-disulfonate potassium salt di-N-hydroxysuccinimide ester) and Cy5 (1,1′-bis(ε-carboxypentyl)-1′ethyl-3,3,3′, 3′-tetramethylindodicarbocyanine-5,5′-disulfonate potassium salt di-N-hydroxysuccinimide ester);

(6) A microarray plate for detecting methylation at cytosine sites in CpG islands in a DNA sample to be tested, on which plate the following oligonucleotides are immobilized:

(a) an oligonucleotide comprising a sequence complementary to a DNA fragment comprising cytosine sites to be tested in the DNA sample, wherein cytosine sites other than the cytosine sites to be tested are substituted with thymines; and

(b) an oligonucleotide comprising a sequence complementary to a DNA fragment comprising cytosine sites to be tested in the DNA sample, wherein all the cytosine sites are substituted with thymines;

(7) A kit for detecting methylation at cytosine sites in CpG islands in a DNA sample to be tested, which comprises:

(a) the microarray plate of (6),

(b) reagents for bisulfite-conversion and/or DNA labeling reagents;

(8) A kit for detecting methylation at cytosine sites in CpG islands in a DNA sample to be tested, which comprises:

(a) an oligonucleotide comprising a sequence complementary to a DNA fragment comprising cytosine sites to be tested in the DNA sample, wherein cytosine sites other than the cytosine sites to be tested are substituted with thymines; and

(b) an oligonucleotide comprising a sequence complementary to a DNA fragment comprising cytosine sites to be tested in the DNA sample, wherein all the cytosine sites are substituted with thymines.

Furthermore, oligonucleotides of the present invention include polynucleotides.

The present invention essentially provides a process wherein the methylation state of cytosine residues within CpG islands is determined by analyzing the signal intensities at defined positions on a microarray device. On a microarray device, each position or site comprises a different capture probe oligonucleotide sequence. Therefore, multiple target molecules can be captured in a multiplex fashion, limited only by the number of capture probe sites available on a microarray device. Those microarray devices developed by CombiMatrix Corporation and marketed through Roche Diagnostics (matriXarray™) for example, can contain up to about 13,000 different sites or an ability to develop a single assay on one chip to evaluate methylation at over 13,000 CpG islands simultaneously.

A minimum of two positions is required on a microarray device to identify the methylation state of each cytosine residue. For example, a sample containing a methylated (M) cytosine residue generates a target molecule that contains a cytosine residue at a specific position, whereas a sample containing an unmethylated (U) cytosine residue generates a target molecule that contains a uracil residue at the specific position. For example, 5′--------------c------------3′ target molecule from methylated (M) sample 5′--------------u------------3′ target molecule from unmethylated (U) sample.

Each of these target molecules is captured at a different position on the microarray device using a capture oligonucleotide probe with a complementary sequence. High stringency conditions during hybridization and wash steps permit a specific capture of a perfectly matched molecule with a specific capture probe, with no capture or minimal capture of molecules that contain a single-base mismatch between the target molecule and oligonucleotide capture probe.

For example, 5′---------------g--------------3′ capture oligonucleotide probe for methylated (M) sample 5′---------------a--------------3′ capture oligonucleotide probe for unmethylated (U) sample

A methylated (M) sample generates a target molecule containing a cytosine residue at the original cytosine position. Its complementary oligonucleotide capture probe on the microarray device containing a guanosine residue captures this target. An unmethylated (U) sample generates a target molecule containing uracil at the original cytosine position. Its complementary oligonucleotide capture probe on the microarray device, wherein the oligo capture probe contains an adenosine residue at the corresponding site, captures this sample.

In a preferred embodiment of microarray hybridization assays, the target molecules/samples are labeled with a fluorescent dye to produce a fluorescent signal that is detected by an optical detection instrument. Alternative means for detection of binding or hybridization includes various electrochemical detection schemes wherein the bound target molecule/oligonucleotide capture probe complex generates an electrode or an electric charge detectable by a nearby electrode. Thus, the methylation state of an unknown sample (test sample) can be determined by measuring binding/hybridization (e.g., the fluorescent signal) at the methylated position (M) or unmethylated position (U) at different known locations on the microarray device.

A single microarray device can contain tens, hundreds or thousands of sites, each with a different capture probe oligonucleotide sequence molecule. The methylation state of tens, hundreds or thousands of CpG islands can be determined on a single microarray at one time. However, since determination of the methylation state of hundreds or thousands of CpG island positions is performed at one time, non-specific or artifact events may interfere with robust determination of methylation state at each relevant position. The present inventive process significantly or completely eliminates the probability of obtaining false positives. This is achieved by incorporation an internal reference sample into the assay.

A reference sample, also known as a control sample, is a nucleic acid sample whose methylation state is known. Existing protocols for multiplex determination of methylation state on arrays require the availability of a separate sample for use as a control or reference (Huang et al., Hum. Mol. Genetics 8:459-70, 1999; Balog et al., Anal Biochem. 309:301-310, 2002). The reference sample is obtained from normal tissue adjacent to a tumor tissue, for example. The methylation state of the reference sample is independently determined before it can be used as a reference or control. Alternatively, a reference sample can be produced by chemical synthesis of DNA representing the region being studied. In another protocol for multiplex determination of methylation state a reference or control sample is not used (Adorjan et al., Nucleic Acid Res. 30(5):e21). In this case, the signal intensities from the methylated probe sequence are compared to the signal intensity of the unmethylated probe sequence. However, non-specific or artifact events may interfere with robust ratio determination of methylation state at each relevant position. In a preferred embodiment of the present invention a DNA sample is analyzed in one fluorescent channel while the same DNA sample is used as a reference sample in another fluorescent channel. The reference sample is prepared from the original sample such that any methylation in the original sample is removed to produce a reference sample that is used as an internal negative control. The only sample required for this embodiment is the DNA sample being tested. No other DNA is required, such as synthetically generated reference DNA or DNA from non-cancerous tissue.

For example, an unknown sample may contain both methylated and unmethylated cytosine residues within CpG islands. When the unknown sample is used as a template in a polymerase chain reaction (PCR) the resultant amplicon contains only unmethylated cytosine. This is due to the fact amplification of the template incorporates unmethylated dCTP that is mixed into the polymerase reaction. The product of this reaction is used as a negative internal control in any hybridization assay.

An unknown sample (test) and a known sample (reference) are mixed together and allowed to hybridize to the microarray device. Since both test and reference samples may hybridize to the same capture probe sequences at a particular site on the microarray device, it will be impossible to determine how much of the signal originated from the test sample and how much of the signal originated from the reference sample, irrespective of the choice of hybridization detection means employed. According to the present inventive process, the test and reference samples are labeled with two different fluorescent dyes so that the signal from each source can be measured separately using wavelength-specific detection of fluorescence. Therefore, using two different fluorescent colors in a 2-color assay. In this manner, the signal intensity of the test sample is measured by detection in a reader channel to look for the first fluorescent dye, and the signal intensity of the reference sample is measured by the detection in a reader channel to look for the second fluorescent dye, wherein the first and second fluorescent dye do not have overlapping emission and excitation wavelengths.

The reference samples can be prepared in a number of different ways for presentation to a microarray device to measure hybridization. For example, a starting material for this methylation assay is genomic DNA. This material is isolated and purified from tissues or cells using a number of existing methods. Purified genomic DNA is prepared for microarray hybridization following the scheme shown in FIG. 2. Roughly equal amounts of genomic DNA are placed in two separate tubes, one for reference sample preparation and one for test sample preparation.

The reference sample is prepared by an initial PCR step (PCR1) using forward (F₁) and reverse (R₁) primers that are designed to anneal to the template DNA at a position outside of the CpG island being tested to form an amplicon. The amplicon that is produced can have a length of approximately 50 base pairs or 500 base pairs to even 1000 base pairs. The amplicon is purified and treated with sodium bisulfite using standard protocols. The treatment converts cytosine residues to deoxyuracil residues since cytosine residues in the reference sample are unmethylated (U) after the first PCR step (PCR1). Deoxyuracil residues behave as thymidine residues in subsequent enzymatic and annealing reactions. One method for the sodium bisulfite conversion step for a methylation assay follows a procedure described in Frommer et al., PNAS 89, 1827-1831. One method is to (1) Dilute DNA (up to 2 μg) with dH₂O to 50 μl (the amount of DNA to be methylated per reaction should be kept constant); (2) Add 5.5 μl 2M NaOH; (3) Incubate at 37° C. for 10 min to denature DNA; (4) Add 30 μl of 10 mM hydroquinone (prepare by adding 55 mg to 50 mL dH₂O); (5) Add 520 μl freshly prepared 3M sodium bisulfite (prepared by adding 1.88 g sodium bisulfite to 5 mL dH₂O—adjust pH to 5.0 with NaOH); (6) Mix well, incubate at about 50° C. for 16 hours; and (7) Clean up DNA (e.g., Qiagen or Promega kit or a reverse phase column e.g., 3M Empore Disk cartridges 4240). If a reverse phase column is used, (a) Add 450 microliters of 10 mM Triethanolamine, 1 mM EDTA, 0.1M Tris pH 7.7; (b) Wash twice with 750 microliters of 10 mM Triethanolamine, 1 mM EDTA, 0.1M Tris pH 7.7; (c) Elute with 50/50 methanol/water+0.3M NaOH; and (d) Speedvac until dry. In addition, continue the process by (8) Resuspend recovered DNA in 50 μl dH₂O, add 5.5 μl of 3M NaOH, incubate at room temperature for 5 min; (9) ETOH precipitate DNA, using a carrier such as glycogen; and (10) store the DNA like RNA (i.e., keep cold, minimize freeze thaws, store at −20° C.).

The converted DNA can now be amplified by PCR. It is important to note that the 2 strands of converted DNA are no longer complimentary, so one has to decide which strand (sense or antisense) to amplify. Primers are designed to amplify fully converted DNA (i.e., all C residues are now T). It should be noted that theoretically, the amount of DNA to be methylated per reaction should be kept constant. Moreover, the bisulfite conversion process is not that efficient as one has often measured 60-80% conversion of unmethylated C's. Further, controls using sss-1 methylase (NEB) can be generated to estimate conversion efficiencies.

The converted genomic DNA product is used as a template in a second PCR step (PCR2) using forward (F₂) and (R₂) primers. Since the sodium bisulfite conversion (SBC) step generated thymidine residues at every cytosine position, F₂ and R₂ primers are designed to anneal to a template that contains thymidine at every cytosine position. The forward primer also contains an RNA polymerase promoter sequence at the 5′ end for T7 polymerase. The purified amplicon from PCR2 is used in an in vitro transcription reaction to generate single-stranded RNA molecules suitable for hybridization to the microarray. Fluorescent dyes, such as Cy3 (1,1′-bis(ε-carboxypentyl)-1′ethyl-3,3,3′,3′-tetramethylindocarbocyanine-5,5′-disulfonate potassium salt di-N-hydroxysuccinimide ester) or Cy5 (1,1′-bis(ε-carboxypentyl)-1′ethyl-3,3,3′,3′-tetramethylindodicarbocyanine-5,5′-disulfonate potassium salt di-N-hydroxysuccinimide ester) (Amersham) are incorporated into the product during transcription. In this example, Cy3 UTP (Amersham Cat# PA53026) is used to fluorescently label the reference target RNA. In the reference sample, all cytosine residues, including methylated (M) and unmethylated (U) cytosine, convert to thymidine during bisulfite conversion. The Cy3 labeled reference target RNA, therefore, contains uracil residues at every cytosine position in the original starting material.

The test sample does not undergo an initial PCR step as does the reference sample. Instead, the test sample DNA is treated directly with sodium bisulfite to convert unmethylated (U) cytosine residues to thymidine. Methylated (M) cytosine residues are not converted and retain their cytosine structure. The resulting product is used as a template in a PCR step (PCR2) using forward (F₂) and reverse (R₂) primers. The primers are designed to anneal to the template DNA at a position outside of the CpG island being tested. The amplicon that is produced can have a length of approximately 50 base pairs or 500 base pairs or even 1000 base pairs.

The same PCR primers are used for the reference sample and the test sample in PCR2. In a preferred embodiment, the forward primer contains an RNA polymerase promoter sequence at the 5′ end for T7 polymerase.

Different PCR primers pairs are required for amplification of each region that is being queried. When several CpG islands are in close proximity, the same pair of PCR primers is used to encompass all CpG islands in the amplicon. When CpG islands are not in close proximity, separate PCR primer pairs are used for each CpG island being queried. When PCR amplification is performed for multiple sites, amplification reactions can be done in a multiplex fashion by combining multiple sets of PCR primers into one reaction mixture yielding multiple sets of amplicons from different regions of the DNA template. PCR primers for multiplex reactions are designed by accurately predicting primer hybridization, evaluating template secondary structure, selecting matching primer pairs, and identifying non-specific primer binding sites. Products from many multiplex reactions are combined to generate pools of amplicons for tens, hundreds or even thousands of methylation sites.

The purified amplicon from PCR2 is used in an in vitro transcription reaction to generate single-stranded RNA molecules. The single-stranded RNA molecules are suitable for hybridization to the microarray. Fluorescent dyes, such as Cy3 (1,1′-bis(ε-carboxypentyl)-1′ethyl-3,3,3′,31-tetramethylindocarbocyanine-5,5′-disulfonate potassium salt di-N-hydroxysuccinimide ester) or Cy5 (1,1′-bis(ε-carboxypentyl)-1′ethyl-3,3,3′,3′-tetramethylindodicarbocyanine-5,5′-disulfonate potassium salt di-N-hydroxysuccinimide ester) are incorporated into the product during transcription. In this example, Cy5 UTP (Amersham Cat# PA55026) is used to fluorescently label the test target RNA. Therefore, in the test sample, all unmethylated (U) cytosine residues convert to thymidine during bisulfite conversion. Methylated (M) cytosine residues do not convert and retain their structure. The first fluorescent dye-labeled single stranded test target RNA contains uracil residues at every cytosine position in the original starting material except for cytosine positions that were methylated. In that case, cytosine residues are present in the test target RNA at every cytosine position in the original starting material. 5′--------------c------------3′ target molecule from methylated (M) sample 5′--------------u------------3′ target molecule from unmethylated (U) sample

The interaction between the target and the oligonucleotide capture probe on the microarray device is able to discriminate between a perfect-match hybrid and single-mismatch hybrid by controlling hybridization conditions. For example, highly stringent hybridization conditions to discriminate between single base pair mismatches would be as follows: a test sample is prepared in 50 ul 6×SSPE, 5× Denhardt's reagent (Sigma), 0.05% Tween 20 and hybridized with a microarray device at 50° C. for 6 hours. The microarray is washed with 300 ul of 6×SSC and 0.05% Tween 20 at 50° C., followed by 300 μl of 2×SSC at 48° C., followed by 300 μl of 1×SSC and finally 300 μl 0.5×SSC both at room temperature.

A perfect-match hybrid generates a fluorescent signal when imaged with a fluorescent microarray optical detection device (such as those manufactured by Axon Instruments, Agilent, Applied Precision and others). The single-mismatch hybrid is thermodynamically unstable and does not form a stable hybrid. Therefore, no fluorescent signal is generated at that position on the microarray device.

A single base difference between a methylated and unmethylated sample is identified on the microarray by the presence or absence of signal at positions containing the complementary sequence to each target molecule in solution. Since the intensity of the fluorescent signal at each position reflects the amount of material in each sample, the state of methylation at each CpG island is determined by measuring the fluorescent signal at a methylation (M) or unmethylation (U) position on the microarray.

The reference sample produces signal at the unemethylated (U) position on the microarray in a second fluorescent probe detection channel. This serves as internal negative control and increases the reliability of results obtained from this assay. If the signal in the first fluorescent probe detection channel (i.e., test sample) is similar to the signal pattern in the second fluorescent probe channel (i.e., reference sample), the test sample is unmethylated at the cytosine position within the CpG island of interest. Conversely, if the signal in the first fluorescent probe detection channel (i.e., test sample) is different from the signal in the second fluorescent probe channel (i.e., reference sample), the test sample is methylated at the cytosine position within the CpG island of interest.

Microarray Design

In a test sample, all or most unmethylated (U) cytosine residues convert to thymidine during bisulfite conversion. Methylated (M) cytosine residues do not convert and retain their structure. The first fluorescent labeled test target RNA contains uracil residues at every cytosine position in the original starting material except for cytosine positions that were methylated. In that case, cytosine residues are present in the test target RNA at every cytosine position in the original starting material.
5′--------------c------------3′ target molecule from methylated (M) sample
5′--------------u------------3′ target molecule from unmethylated (U) sample

The interaction between the target and the capture probe on the microarray is able to discriminate between a perfect-match hybrid and single-mismatch hybrid under appropriate high stringency hybridization conditions. Appropriate high stringency hybridization conditions include, for example, hybridization in 50 μl 6×SSPE, 5× Denhardt's reagent (Sigma), 0.05% Tween 20 at 50° C. for 6 hours, followed by washing with 300 μl of 6×SSC and 0.05% Tween 20 at 50° C., followed by washing with 300 μl of 2×SSC at 48° C., followed by washing with 300 μl of 1×SSC and finally with 300 μl 0.5×SSC both at room temperature.

A more preferable high stringent condition is conducting hybridization at 55° C. in the solution above instead of 50° C. However, other than temperature, several factors, such as salt concentration, can influence the stringency of hybridization and one skilled in the art can suitably select the factors to accomplish a similar stringency.

The perfect-match hybrid on the microarray generates a fluorescent signal when imaged with an optical detection device. Alternatively other common microarray detection technologies can be used, such as electrochemical detection on microarray devices have electrodes and electronic signal hybridization detection technologies. The single-mismatch hybrid is thermodynamically unstable and does not form a stable hybrid. Therefore, no detectable signal is generated at that position on the microarray device.

The microarray is designed to have at least one capture probe for the methylated target (M) and one capture for the unmethylated target (U). Example sequences are shown below:

Methylated Sample Capture Probe Sequence (M)

5′-uauuuuuuuagguagcggguaguaguuguuu-3′ target sequence [SEQ ID NO. 1]
3′-auaaaaaaauccaucgcccaucaucaacaaa-5′ capture probe sequence (3′-5′) [SEQ ID NO. 2]
5′-aaacaacuacuacccgcuaccuaaaaaaaua-3′ capture probe sequence (5′-3′) [SEQ ID NO. 3]
   Unmethylated Sample Capture Probe Sequence (U)
5′-uauuuuuuuagguaguggguaguaguuguuu-3′ target sequence [SEQ ID NO. 4]
3′-auaaaaaaauccaucacccaucaucaacaaa-5′ capture probe sequence (3′-5′) [SEQ ID NO. 5]
5′-aaacaacuacuacccacuaccuaaaaaaaua-3′ capture probe sequence (5′-3′) [SEQ ID NO. 6]

Multiple methylation assays are performed at the same time on one microarray. Here, methylation assays ‘1 through . . . ’ are performed in parallel. Each methylation assay has its own pair of methylated (M) and unmethylated (U) capture probes on the microarray.

Since the reference sample undergoes an initial PCR step, only unmethylated cytosine is present in the DNA amplicon prior to transcription. The reference sample represents signal from an unmethylated source. The test sample is not treated with an initial PCR step and the methylation state of the cytosine residues is retained prior to sodium bisulfite conversion. The methylation state is determined by comparing the signal intensities of the test sample in a second fluorescent probe channel to the test sample in a first fluorescent probe channel. If there is equal signal intensity in both the first and second channels the test sample is unmethylated at the specific CpG island of interest. If there is signal intensity in the first fluorescent probe channel but not in the second fluorescent probe channel, the test sample is methylated (see FIG. 4).

FIG. 5 shows results from a multiplexed two-color methylation detection assay performed on a microarray. Multiple methylation sites (CpG islands) are detected at one time on the same microarray (1 to . . . ). The ‘M’ position contains capture probe sequences for a methylated sample. The ‘M’ capture probe contains a guanosine residue at the cytosine position of the original cytosine in CpG island and adenosine residues at all other cytosine positions. The ‘U’ position contains capture probe sequences for an unmethylated sample. The ‘U’ capture probe contains adenosine residues at all cytosine positions in the original sample.

In the data illustrated in FIG. 4, the second fluorescent probe signals obtained from the reference sample represent the pattern obtained by an unmethylated sample. Very little or no signal is detected at the Methylation probe (M) and a large amount of signal is detected at the Unmethylation probe (U). This pattern establishes the reference signal for an unmethylated sample. The first fluorescent probe signals represent the methylation state of the test sample. The microarray determines the methylation state of hundreds or thousands of CpG islands in a multiplex fashion. The signal at position 1 shows the results from CpG island number 1. The signal at position 2 shows the results from CpG island number 2, and so on. The signal at position 1 displays strong fluorescent signal at the Unmethylation probe (U) and very little or no signal at the Methylation probe (M). This pattern is similar to the signal pattern in the reference sample for position 1. The interpretation is made that CpG island number 1 is unmethylated (U).

Further, in FIG. 4, the signal at position 2 displays strong fluorescent signal at both the Unmethylation probe (U) and the Methylation probe (M). This pattern is different from the signal pattern in the reference sample for position 2. Since both Methylation and Unmethylation probes generate fluorescent signal in the test sample, the interpretation is made that CpG island number 2 has both a methylated and unmethylated allele (M/U). The signal at position 3 displays strong fluorescent signal at the Methylation probe (M) and very little or no signal at the Unmethylation probe (U). This pattern is different to the pattern in the reference sample for position 3. The interpretation is made that CpG island number 3 is methylated (M).

By following the interpretation logic illustrated with the data shown in FIG. 4, the methylation state of hundreds or thousand of CpG islands can be determined on a single microarray at one time. Once the fluorescent signal is obtained, the process of interpretation, or calling the methylation state, can be performed using suitable computers and software algorithms. This permits rapid interpretation of assay results with the need for human intervention.

Capture Probe Library Screening

Methylation of cytosine residues generally occur within CpG dinucleotide positions. Computer algorithms that scan through known regions of the genome predict sites where methylation of cytosine may occur (http://www.uscnorris.com/cpgislands, Takai and Jones, Proc. Natl. Acad. Sci. 19; 99(6):3740-5, 2002). Additionally, computer databases store DNA methylation sites and allow searching and retrieval of DNA sequences around these sites (http://www.methdb.net/).

The microarrays are composed of hundreds or thousands of different sequence oligonucleotide capture probes to specifically capture target molecules. The target molecules contain either a cytosine residue at the original methylation site if the sample is methylated, or a uracil residue if the sample is unmethylated.
5′--------------c------------3′ target molecule from methylated (M) sample
3′--------------g------------5′ capture probe for methylated (M) sample
5′--------------u------------3′ target molecule from unmethylated (U) sample
3′--------------a------------5′ capture probe for unmethylated (U) sample

Specific hybridization of the target molecule to its complementary capture probe is required for robust assay performance. Hybridization conditions are designed so that a single base mismatch between a capture probe and target molecule does not form a stable hybrid. For example, hybridization is performed in 50 μl 6×SSPE, 5× Denhardt's reagent (Sigma), 0.05% Tween 20 at 50° C. for 6 hours. The array is washed with 300 μl of 6×SSC and 0.05% Tween 20 at 50° C., followed by 300 μl of 2×SSC at 48° C., followed by 300 μl of 1×SSC and finally 300 μl 0.5×SSC both at room temperature.

It is also highly desirable to screen thorough a large number of capture probes at one time. High throughput screening of tens or hundreds of different capture probes against the same target permits rapid and cost-effective development of validated probes sets.

A single-base mismatch placed a different positions along a capture probe can have significant impact on the performance of the capture probe and in its ability to discriminate between a perfectly matched or single base mismatched target. It is desirable to rapidly and effectively screen a library of hundreds or thousands of different capture probes to identify the most reliable sequence. For example, a library of sequences is generated by moving the position of the mismatch sequence (methylation position) along the capture probe sequence. Capture probe for methylated (M) sample:

5′--g------------------------3′
5′----g----------------------3′
5′------g--------------------3′
5′--------g------------------3′
5′-----------g---------------3′
5′-------------g-------------3′
5′---------------g-----------3′
5′-----------------g---------3′
5′-------------------g-------3′
5′---------------------g-----3′
5′-----------------------g---3′

In addition, a library of sequences is generated by increasing the length of the capture probe sequence. Capture probe for methylated (M) sample:

5′-----g-----3′
5′------g------3′
5′-------g-------3′
5′--------g--------3′
5′---------g---------3′
5′----------g----------3′
5′-----------g-----------3′
5′------------g------------3′
5′-------------g-------------3′

By combining the probe length and mismatch position, hundreds or even thousands of different probes are designed for a single CpG island position. The semiconductor based microarray system rapidly synthesizes all probes at one time and the entire library is empirically tested by a screening assay. Only probes with desired performance are selected and used in the final assay.

The microarray device of the present invention refers to a device in which oligonucleotides and such are array-immobilized on a plate, and normally refers to those having nucleotides placed on a plate surface such as glass and silicon. The microarray of the present invention is not limited to so called spotted microarrays. High-density arrays constructed by synthesizing various polynucleotides at once on plates are also called “DNA chips”. Such “chips” on which oligonucleotides are synthesized on plates are also included in the microarray device of the present invention.

In the present invention, the term “plate” refers to a material in the form of a sheet on which nucleotides can be immobilized. In some cases, the microarray device itself is referred to as a plate. Namely, a plate with immobilized oligonucleotides is simply referred to as a “plate”. There are no particular limitations on the plate of the present invention as long as nucleotides can be immobilized on it, and plates (for example, glass and silicon) generally used for microarray technology can be preferably used.

In general, a microarray comprising thousands of polynucleotides spotted onto a plate (the step of immobilizing polynucleotides onto a plate is also called “printing”) at high density. Normally, these nucleotides are spotted (printed) onto the surface of a non-porous plate. The plate generally has a glass surface, but a porous membrane such as nitrocellulose membrane may also be used. In a polynucleotide array, polynucleotides can be synthesized in situ. For example, in situ synthesis methods, such as photolithographic technology (Affymetrix) and ink jet technology for immobilizing chemical substances (Rosetta Inpharmatics), are already known, and either technology can be used for constructing the plates of the present invention. In the present invention, “immobilization” onto a plate includes the meaning of the so called “synthesis”. Those skilled in the art can usually use a commercially available device that allows high-density spotting (printing) to construct, for example, microarrays comprising 10,000 or more kinds of spots (prints) on a slide glass as necessary in the laboratory.

In the present invention, polynucleotides can be immobilized onto a plate after they are artificially synthesized. In this case, the polynucleotides can be synthesized by standard methods well known in the art, for example by using a commercially available automatic DNA synthesizer.

A preferred embodiment of the plate of this invention is a microarray plate (device) for use in the detection process of this invention, which has a plurality of oligonucleotide-capturing probes designed to hybridize to CpG island sites of DNA samples as bisulfite-converted and non-converted forms.

Furthermore, in another embodiment, the plate of this invention is a microarray plate for detecting methylation of cytosine sites in CpG islands in the DNA sample to be tested, on which plate the following oligonucleotides are immobilized:

(a) an oligonucleotide comprising a sequence complementary to a DNA fragment comprising cytosine sites to be tested in the DNA sample, wherein cytosine sites other than the cytosine sites to be tested are substituted with thymines; and

(b) an oligonucleotide comprising a sequence complementary to a DNA fragment comprising cytosine sites to be tested in the DNA sample, wherein all the cytosine sites are substituted with thymines.

The oligonucleotides of (a) and (b) are used as capture probes for methylating samples in the process of this invention.

Furthermore, the various capture probes described in the present invention can be used as oligonucleotides to be immobilized onto the plates of this invention.

Furthermore, the present invention provides a kit to be used for the detection process of this invention. More specifically, the present invention provides a kit for detecting methylation of cytosine sites in CpG islands in a DNA sample to be tested. A preferred embodiment of the kit of this invention is, for example, a kit comprising at least one of (a) and (b) described below:

(a) the microarray plate of the present invention,

(b) reagents for bisulfite-conversion and/or DNA labeling reagents.

An example of the DNA labeling reagents is the aforementioned fluorescent labeling substance.

Furthermore, in another embodiment, the kit of the present invention includes a kit comprising at least one of (a) and (b) described below.

(a) an oligonucleotide comprising a sequence complementary to a DNA fragment comprising cytosine sites to be tested in the DNA sample, wherein cytosine sites other than the cytosine sites to be tested are substituted with thymines; and

(b) an oligonucleotide comprising a sequence complementary to a DNA fragment comprising cytosine sites to be tested in the DNA sample, wherein all the cytosine sites are substituted with thymines.

One skilled in the art can use the oligonucleotides contained in the kit to produce a plate for microarray of this invention appropriately using conventional methods. Plates thus produced are also included in the present invention.

PCR primers used in the method of this invention, positive and negative standard samples (control samples), instructions indicating the method of use of the kit, and such, may be packaged with the kit of this invention.

### BRIEF DESCRIPTION OF DRAWINGS

FIG. 1 shows a schematic of various prior methylation detection processes, some of which are described in the Background section. The present invention can also be added to this scheme as bisulfite based.

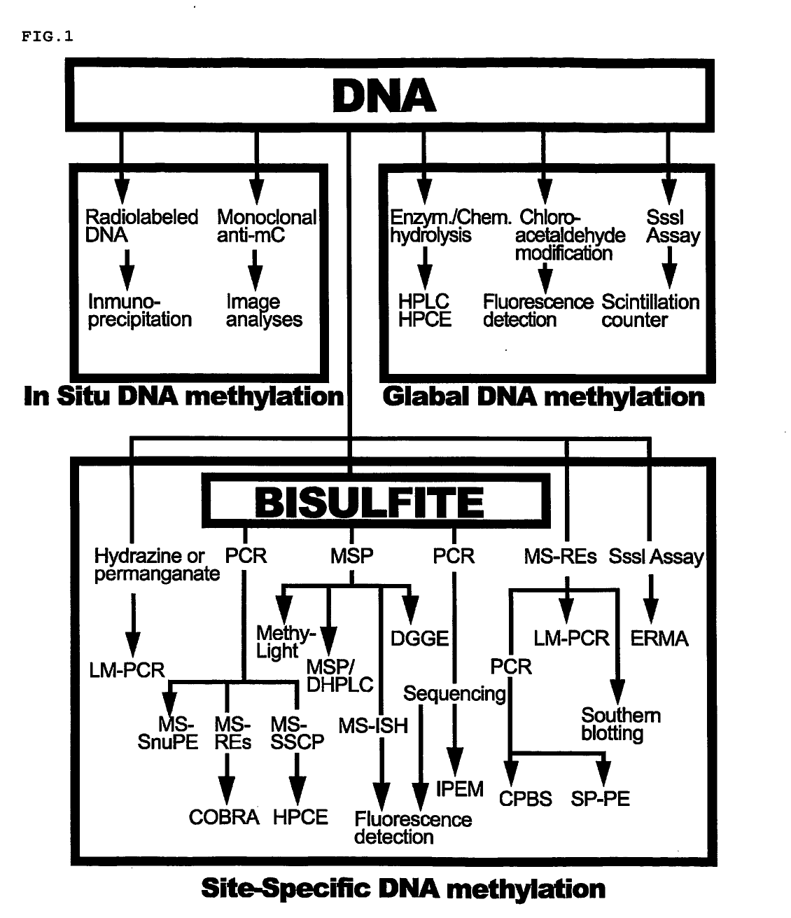

FIG. 2 shows a schematic of a preferred embodiment of the inventive process beginning with DNA (A) and being divided into two arms. The left arm first PCR amplifies the sample (B), then performs bisulfite conversion and then labeling with a second fluorescent probe (Cy3). The right arm first bisulfite converts the sample (C), then PCR amplifies, then labels with a first fluorescent probe (Cy5) and finally both samples (B+C) are hybridized onto a DNA microarray device for detection in a two-color fluorescent imaging reaction.

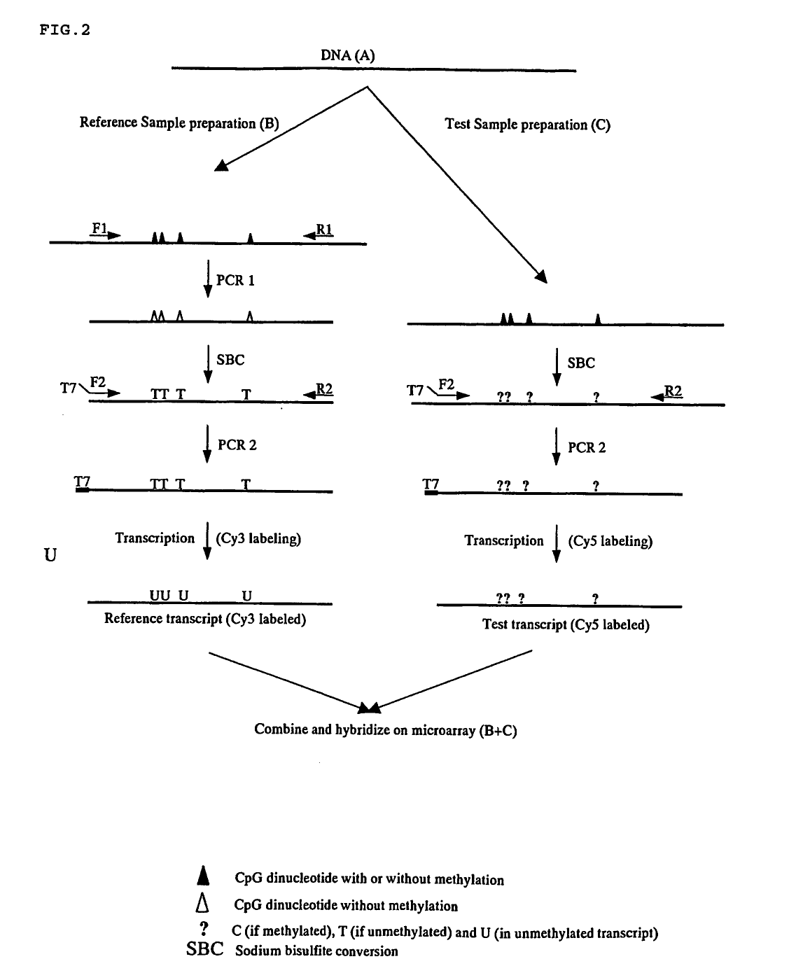

FIG. 3 shows a microarray device layout pattern for studying a large set of CpG island methylation sites in parallel. A minimum of two features or microarray sites is required on the microarray device for each methylation site that is queried. One feature of the microarray device probes for the presence of methylated cytosine and a second feature of the microarray device probes for the presence of unmethylated cytosine in a sample.

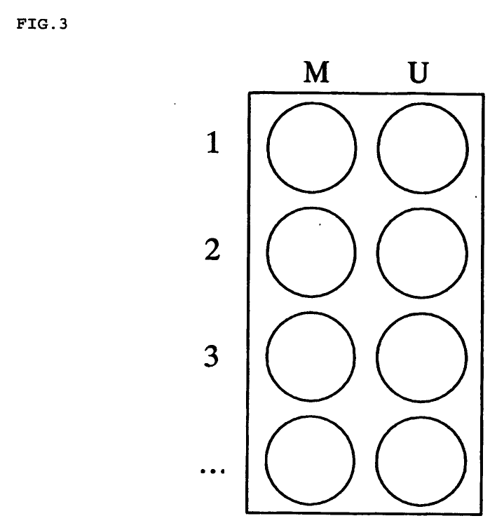

FIG. 4 shows results from a multiplexed two-color methylation detection assay performed on a microarray. Multiple methylation sites (CpG islands) are detected at one time on the same microarray. The ‘M’ position contains capture probe sequences for a methylated sample. The ‘M’ capture probe contains a guanosine residue at the cytosine position of the original cytosine in CpG island and adenosine residues at all other cytosine positions. The ‘U’ position contains capture probe sequences for an unmethylated sample. The ‘U’ capture probe contains adenosine residues at all cytosine positions in the original sample.

FIG. 5 shows hybridization discrimination between a perfectly-matched 15mer DNA target and single-mismatch 15mer DNA target hybridized under high stringency conditions. Capture probes were designed such that the single mismatch position shifted from the fifth position of the capture probe to the eleventh position. Spot intensity ratio between match and mismatch samples indicates that maximal discrimination was obtained when the mismatch was positioned at the center of the capture probe.

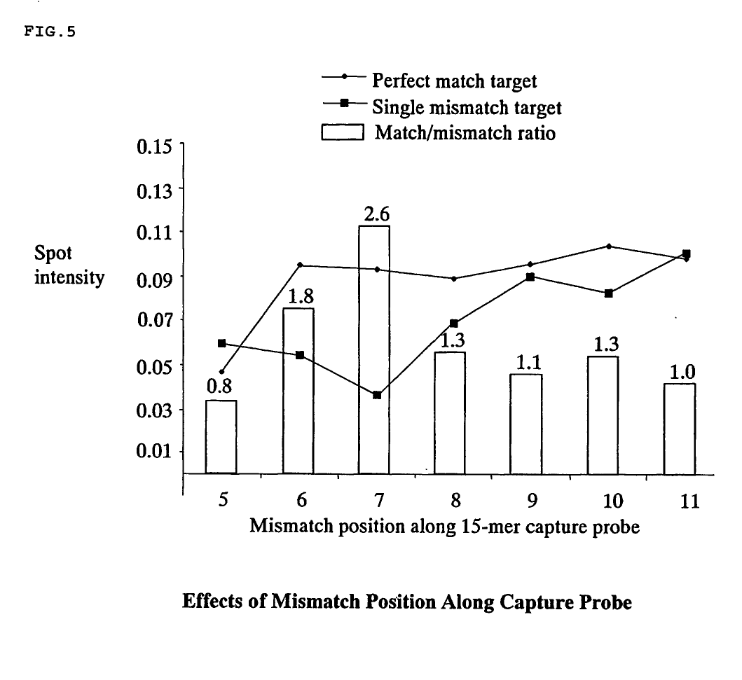

FIG. 6 shows preparation of a reference sample for the sequence region being studied. The sample being tested serves as its own internal reference control according to the inventive process. Amplification of DNA by the first PCR step strips all methylation information from the sample. During PCR1, methylated and unmethylated cytosine residues produce unmethylated cytosine residues in the amplicon. The amplicon undergoes bisulfite conversion and cytosine residues are converted to deoxyuracil, which behave as thymidine residues in further enzymatic and annealing reactions. The bisulfite-converted product undergoes a second PCR step to add an upstream transcriptional promoter. The final Cy3-labeld transcript is used as a reference sample that generates the unmethylated signal pattern on the microarray.

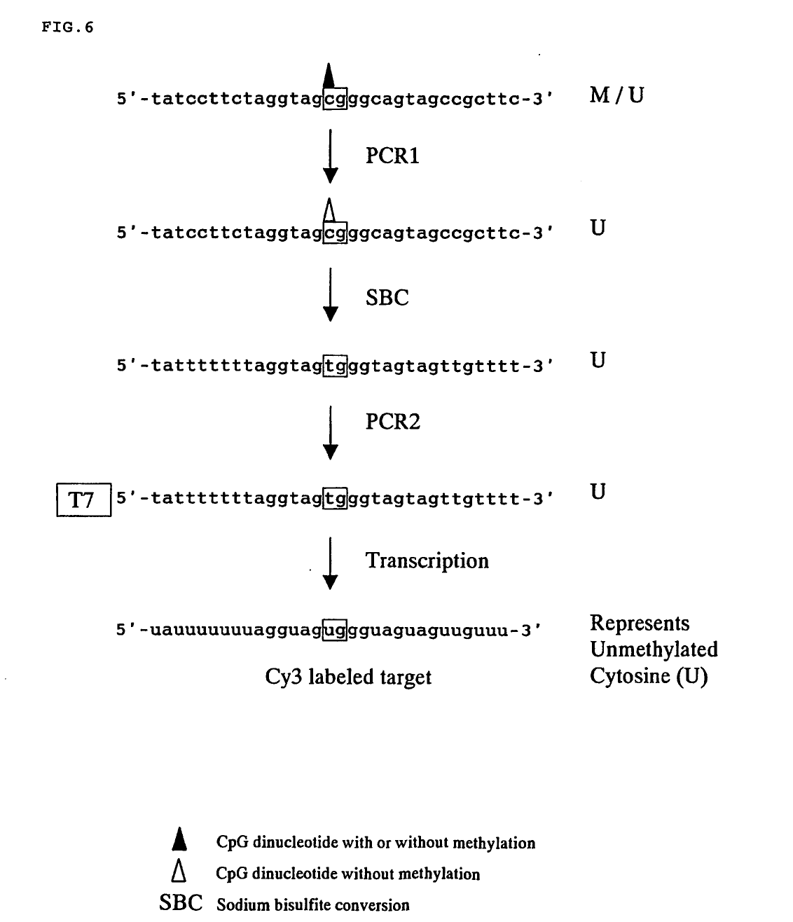

FIG. 7 shows preparation of a test sample for the region being studied. The sample is treated with sodium bisulfite to convert unmethylated cytosine residues to deoxyuracil, which behave as thymidine residues in further enzymatic and annealing reactions. Methylated cytosine residues are protected during the conversion and retain their original cytosine structure. The bisulfite-converted products undergo a PCR step to add an upstream transcriptional promoter. The final Cy5-labeld transcript is used as the test sample to generate methylated or unmethylated signals on the microarray that reflect the original methylation state of the sample being tested.

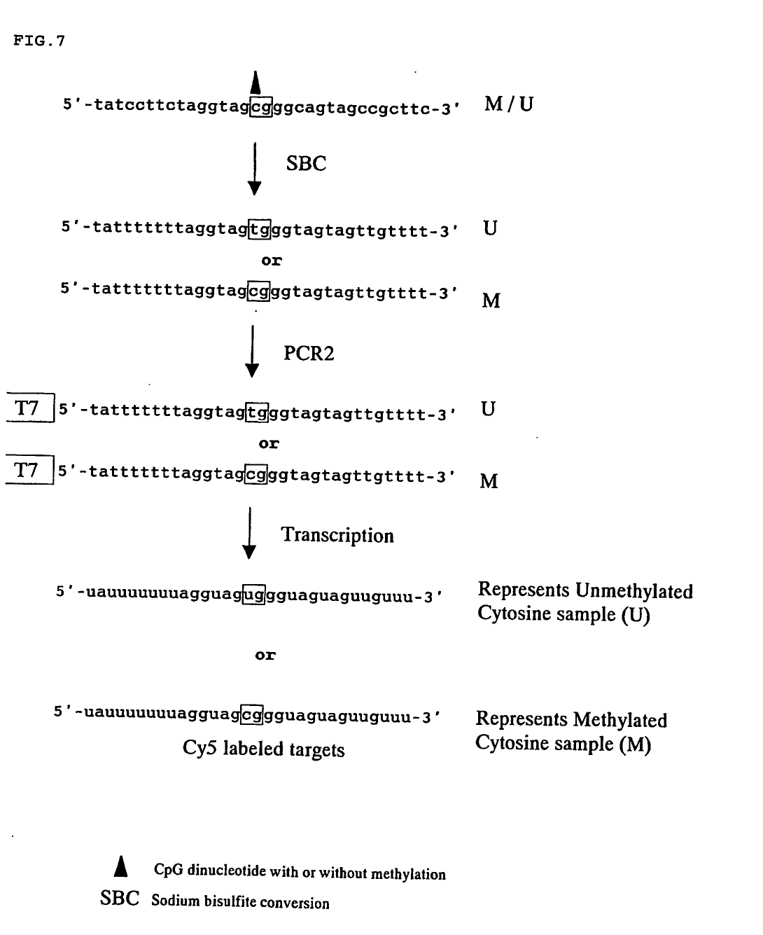

### BEST MODE FOR CARRYING OUT THE INVENTION

### Example 1

This example illustrates a multiplex methylation assay using the inventive procedure and a microarray device from CombiMatrix Corp. (made using in situ synthesis with an electrochemical process).

Sample Collection and DNA Purification.

Homo sapiens DNA mismatch repair (hMLH1) gene (GenBank ACCESSION U83845), a human primary colon carcinoma, cell line SW480 (purchased from ATCC; http://www.atcc.org/), and 293T (purchased from ATCC) were used. DNA was purified as follows. The cells were cultivated, and were collected from dishes. The collected cells were washed in PBS three times. The washed cells were centrifugated, and stored at −80° C. Cell aggregates were suspended in reaction buffer, and were digested by proteinase K. After digestion, the aqueous phases were extracted with, a 1:1 mixture of equilibrated phenol and chloroform. DNA was recovered by 70% ethanol precipitation, and was suspended in pure water or TE (1.0 mg/mL).

Sequence being investigated:

| | |
|---|---|
| 5′ atcacctcagcagaggcacacaagcccggttccggcatctctgctcctattggctggatatt | [SEQ ID NO. 7] |
| tcgtattccccgagctcctaaaaacgaaccaataggaagagcggacagcgatctctaacgcgca | |
| agcgcatatccttctaggtagcgggcagtagccgcttcagggagggacgaagagacccagcaac | |
| ccacagagttgagaaatttgactggcattcaagctgtccaatcaatagctgccgctgaagggtg | |
| gggctggatggcttaagctacagctgaaggaagaacgtgagcacgaggcactgaggtgattggc | |
| tgaaggcacttccgttgagcatctagacgtttccttggctcttctggcgccaaaatg-3′ | |

Genomic DNA (100 ng) was aliquoted into two tubes, one labeled reference sample and the other labeled test sample.

Test Sample Preparation

The Reference sample undergoes a PCR amplification with the following two primers:

| | |
|---|---|
| Forward primer 1 (F1): | |
| 5′-atcacctcagcagaggcacac-3′ | [SEQ ID NO. 8] |
| Reverse primer 1 (R1): | |
| 5′-tttggcgccagaagagccaag-3′ | [SEQ ID NO. 9] |

PCR amplification was performed in a total volume of 50 μl containing 1×PCR Gold® buffer (Applied Biosystems, Foster City, Calif.), 2 mM MgCl₂, 0.2 mM deoxynucleotide triphosphates mixture (USB), 10 pmol forward primer (F1, SEQ ID NO. 8), 10 pmol reverse primer (R1, SEQ ID NO. 9), 2 U Amplitaq Gold DNA® polymerase (ABI), and 100 ng template DNA. Reaction conditions were as follows: 95° C. for 10 minutes, and 39 cycles of 92° C. for 30 seconds, 57° C. for 30 seconds, and 70° C. for 30 seconds, with a final elongation for 7 minutes at 70° C. The PCR products were analyzed by gel electrophoresis using a 2.5% agarose gel, stained with ethidium bromide and visualized under UV illumination with a digital imaging system (NucleoTech). Amplicons were purified using QIAquick® PCR purification kits (Qiagen) following manufacturers protocol.

Sodium Bisulfite Conversion (SBC)

One microgram purified amplicon was diluted in 50 μl of distilled water and denatured by addition of 1 μl 10 M sodium hydroxide to a final concentration of 0.2 M and incubated for 10 minutes at 37° C. After incubation, 30 μl 10 mM hydorguinone (Sigma) and 520 μl of 3M sodium bisulfite (Sigma) at pH 5.0 were added. The solution was incubated at 53° C. for 18-20 hours.

DNA was purified by QIAquick® purification kits (Qiagen) following the manufacturer's protocol. The DNA was desulfonated with 0.3 M sodium hydroxide for 10 minutes at room temperature, neutralized with 17 μl of 10 M ammonium acetate (Ambion) and then precipitated in 100% ethanol at −80° C. overnight.

The second PCR amplification of the reference sample used the following primers:

[SEQ ID NO. 10]

Forward primer 2 (F₂):
5′-taatacgactcactatagggattattttagtagaggtatat-3′

[SEQ ID NO. 11]

Reverse primer 1 (R₂):
5′-tttggtgttagaagagttaag-3′

Amplification was performed in a total volume of 50 μl containing 1×PCR Gold® buffer (Applied Biosystems, Foster City, Calif.), 2 mM MgCl₂, 0.2 mM deoxynucleotide triphosphates mixture (USB), 10 pmol forward primer 2 (F₂, SEQ ID NO. 10), 10 pmol reverse primer (R₂, SEQ ID NO. 11), 2 U Amplitaq Gold DNA® polymerase (Applied Biosystems, Foster City, Calif.), and 100 ng template DNA. Reaction conditions were as follows: 95° C. for 10 minutes, and 39 cycles of 92° C. for 30 seconds, 57° C. for 30 seconds, and 70° C. for 30 seconds, with a final elongation for 7 minutes at 70° C. The PCR products were analyzed by gel electrophoresis using a 2.5% agarose gel, stained with ethidium bromide and visualized under UV illumination with a digital imaging system (NucleoTech). Amplicons were purified using QIAquick® PCR purification kits (Qiagen) following manufacturers protocol.

Transcription

One microgram purified amplicon containing T7 promoter sequence was transcribed in vitro in a total volume of 20 μl using MEGAscript® Kits (Ambion) following the manufacturers protocol with the addition of 5 μl of 10 mM Cy3 UTP (Amersham). The transcripts were purified using RNeasy® purification kits (Qiagen) following the manufacturer's protocol.

Test Sample Preparation

The test sample is first converted in a sodium bisulfite conversion (SBC) step. Briefly, one microgram genomic DNA was diluted in 50 μl of distilled water and denatured by addition of 1 μl 10 M sodium hydroxide to a final concentration of 0.2 M and incubated for 10 minutes at 37° C. After incubation, 30 μl 10 mM hydorquinone (Sigma) and 520 μl of 3M sodium bisulfite (Sigma) at pH 5.0 were added. The solution was incubated at 53° C. for 18-20 hours.

The DNA was desulfonated with 0.3 M sodium hydroxide for 10 minutes at room temperature, neutralized with 17 μl of 10 M ammonium acetate (Ambion) and then precipitated in 100% ethanol at −80° C. overnight.

The test sample genomic DNA was then amplified by a PCR 2 amplification using the following primers:

[SEQ ID NO. 12]

Forward primer 2 (F₂):
5′-taatacgactcactatagggattattttagtagaggtatat-3′

[SEQ ID NO. 13]

Reverse primer 1 (R₂):
5′-tttggtgttagaagagttaag-3′

PCR amplification was performed in a total volume of 50 μl containing 1×PCR Gold buffer (Applied Biosystems, Foster City, Calif.), 2 mM MgCl₂, 0.2 mM deoxynucleotide triphosphates mixture (USB), 10 pmol forward primer 2 (F₂), 10 pmol reverse primer (R₂), 2 U Amplitaq Gold DNA polymerase (Applied Biosystems, Foster City, Calif.), and 100 ng template DNA. Reaction conditions were as follows: 95° C. for 10 minutes, and 39 cycles of 92° C. for 30 seconds, 57° C. for 30 seconds, and 70° C. for 30 seconds, with a final elongation for 7 minutes at 70° C. The PCR products were analyzed by gel electrophoresis using a 2.5% agarose gel, stained with ethidium bromide and visualized under UV illumination with a digital imaging system (NucleoTech). Amplicons were purified using QIAquick PCR purification kits (Qiagen) following manufacturer's protocol.

The test sample was then subject to transcription. Briefly, one microgram of purified amplicon containing T7 promoter sequence was transcribed in vitro in a total volume of 20 μl using MEGAscript Kits® (Ambion) following the manufacturers protocol with the addition of 5 μl of 10 mM Cy5 UTP (Amersham). The transcripts were purified using RNeasy® purification kits (Qiagen) following the manufacturer's protocol.

Hybridization and Wash

The reference transcript (4 μg) was labeled with a second fluorescent dye, preferably Cy3 and combined with the test transcript (4 μg) labeled with a first fluorescent dye, preferably Cy5 in 50 μl 6×SSPE, 5× Denhardt's reagent (Sigma), 0.05% Tween 20. The mixture was hybridized with a microarray device at 50° C. for 6 hours. The microarray was washed with 300 μl of 6×SSC and 0.05% Tween 20 at 50° C., followed by 300 μl of 2×SSC at 48° C., followed by 300 μl of 1×SSC and finally 300 μl 0.5×SSC both at room temperature.

Microarray Device

The microarray is designed to have at least one capture probe for the methylated target (M) and one capture for the unmethylated target (U).

Methylated Sample Capture Probe Sequence (M)

5′-uauuuuuuuagguagcggguaguaguuguuu-3′ target sequence [SEQ ID NO. 1]
3′-auaaaaaaauccaucgcccaucaucaacaaa-5′ capture probe sequence (3′-5′) [SEQ ID NO. 2]
5′-aaacaacuacuacccgcuaccuaaaaaaaua-3′ capture probe sequence (5′-3′) [SEQ ID NO. 3]
   Unmethylated Sample Capture Probe Sequence (U)
5′-uauuuuuuuagguaguggguaguaguuguuu-3′ target sequence [SEQ ID NO. 4]
3′-auaaaaaaauccaucacccaucaucaacaaa-5′c apture probe sequence (3′-5′) [SEQ ID NO. 5]
5′-aaacaacuacuacccacuaccuaaaaaaaua-3′ capture probe sequence (5′-3′) [SEQ ID NO. 6]
   Imaging and Data Analysis

After the final wash step, the microarray was imaged using an optical detection instrument having a CCD camera (arrayWoRx Biochip Reader, Applied Precision). Two images were captured from each microarray corresponding to the emission wavelength of each fluorescent dye. The images were saved on a microcomputer and analyzed following manufacturer's instructions (softWoRx Tracker, Applied Precision). The fluorescent intensity at each position on the microarray was quantified and saved as a spreadsheet containing probe sequence and position information as well as fluorescent intensity of each dye.

Intensity data was analyzed, for example, by calculating the ratio of signal intensity between the test sample having the first fluorescent dye and the reference sample having the second fluorescent dye. In this manner, the methylation state of cytosine residues within a CpG island is determined.

Ratio analysis of data for probe signal and reference signal:

$${R}_{m} = \frac{( \frac{{M}_{test}}{{M}_{ref}} )}{( \frac{{U}_{test}}{{U}_{ref}} )}$$

### EXAMPLE 2

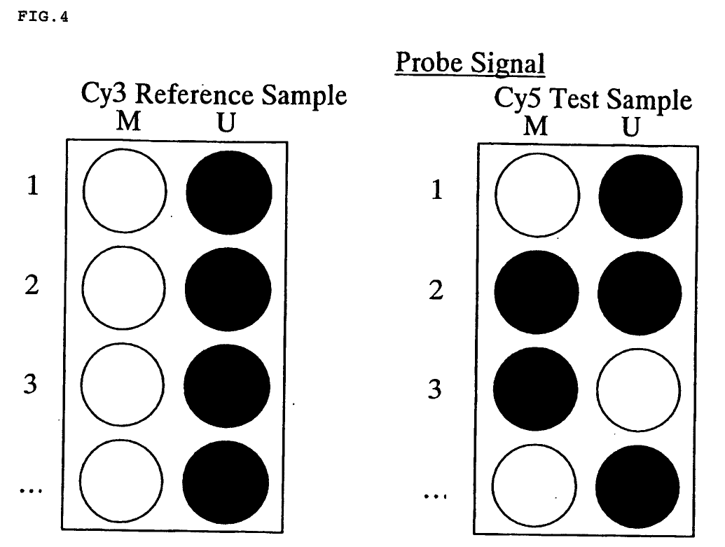

This example performs the analysis of multiple CpG islands simultaneously using the procedure described in Example 1 for a single CpG island methylation determination. Signal intensities shown in FIG. 4 are analyzed by calculating the ratios for each probe in both the test and reference signal channels. For example, the first row of the microarray shown in FIG. 4 is designed to assess the methylation state of region 1 in a sample. The second row is designed to assess the methylation state in region 2 of the same sample, and so on. Each region in the sample may be methylated (M) or unmethylated (U). The sample is prepared as describe in Example 1. The sample being tested is used as its own internal reference control. Preparation of the reference sample removes any methylation that may have been present. The reference target is labeled with Cy3 fluorescent dye, for example, and its signal appears in the Cy3 detection channel. The test sample is processed so that methylation is retained during preparation. The test sample is labeled with Cy5 fluorescent dye, for example, and its signal appears in the Cy5 detection channel. FIG. 4 shows signal intensities appearing in each channel in black.

The results for region 1 are shown in the first row of the microarray. The reference sample appears with fluorescent signal at the unmethylated probe (U) position as expected. The test sample also appears with signal at the unmethylated probe (U) position, similar to the reference sample. By calculating these results using the formula provided in Example 1, the interpretation is made that the sample is unmethylated (U) in region 1.

The results for region 2 are shown in the second row of the microarray. The reference sample appears with fluorescent signal at the unmethylated probe (U) position as expected. The test sample appears with signal at both the unmethylated probe (U) position and the methylated (M) probe position. By calculating these results using the formula provided in Example 1, the interpretation is made that the sample contains both methylated (M) and unmethylated (U) cytosine in region 2 in approximate equal proportions. This occurs when, for example, one allele in the sample is methylated and the other allele is unmethylated. This may also occur in a heterogeneous cell population where approximately one half of the cells are methylated in region 1.

The results for region 3 are shown in the third row of the microarray. The reference sample appears with fluorescent signal at the unmethylated probe (U) position as expected. The test sample appears with signal only at the methylated probe (M) position. By calculating these results using the formula provided in Example 1, the interpretation is made that the sample is entirely methylated (M) in region 3.

Microarrays containing tens, or hundreds, or even thousands positions can be used to determine the methylation state of tens, hundreds or even thousands of different CpG island regions within the same sample and in parallel.

1. A process that simultaneously detects methylation at multiple CpG island sites using a reference sample obtained from a sample to be tested, wherein the process is a nucleic acid methylation detection process that uses an internal reference sample and comprises the steps of:
using a DNA sample for analysis, that is divided into a first DNA sample to be tested and a second DNA sample to be the internal reference, to amplify the second DNA sample such that methylcytosine residues are amplified as unmethylated cytosine residues;
converting the unmethylated cytosine residues to deoxyuracil residues in both the first DNA sample and the second DNA sample;
using a first fluorescent marker and a second fluorescent marker having non-overlapping fluorescent excitation and fluorescent emission spectra to label the first DNA sample with the first fluorescent marker and to label the second DNA sample with the second fluorescent marker; and
hybridizing the first DNA sample and the second DNA sample onto a microarray device having a plurality of oligonucleotide capture probes designed to hybridize to CpG island sites of the DNA sample as converted and non-converted forms.

2. A process that simultaneously detects methylation at a large number of CpG island sites using a reference sample obtained from a sample to be tested, comprising:
(a) providing a DNA sample for analysis;
(b) dividing the DNA sample into a first DNA sample and a second DNA sample, whereby the first sample will become a test sample and the second sample will become an internal reference sample;
(c) amplifying the second DNA sample by a nucleic acid amplification process such that methylcytosine residues are amplified as unmethylated cytosine residues;
(d) bisulfite conversion of unmethylated cytosine residues into deoxyuracil residues in both the amplified first DNA sample and the second DNA sample;
(e) amplifying the converted first DNA sample and the converted second DNA sample;
(f) labeling the bisulfite-converted second DNA sample with a second fluorescent marker and the bisulfite-converted first DNA sample with a first fluorescent marker, wherein the first and second fluorescent markers have non-overlapping fluorescent excitation and emission spectra; and
(g) hybridizing the first DNA sample and the second DNA sample onto a microarray device having a plurality of oligonucleotide capture probes designed to hybridize to CpG island sites of the DNA sample as converted and non-converted by bisulfite.

3. The process of claim 1, wherein the amplification technique employed is PCR (polymerase chain reaction).

4. The process of claim 1, wherein the hybridization conditions are highly stringent conditions.

5. The process of claim 1, wherein the non-overlapping fluorescent labels are Cy3, (1,1′-bis (ε-carboxypentyl)-1′ethyl-3,3,3′,3′-tetramethylindocarbocyanine-5,5′-disulfonate potassium salt di-N-hydroxysuccinimide ester) and Cy5 (1,1′-bis(ε-carboxypentyl)-1′ethyl-3,3,3′,3′-tetramethylindodicarbocyanine-5,5′-disulfonate potassium salt di-N-hydroxysuccinimide ester).

6. A microarray plate for detecting methylation at cytosine sites in CpG islands in a DNA sample to be tested, on which plate the following oligonucleotides are immobilized:
(a) an oligonucleotide comprising a sequence complementary to a DNA fragment comprising cytosine sites to be tested in the DNA sample, wherein cytosine sites other than the cytosine sites to be tested are substituted with thymines; and
(b) an oligonucleotide comprising a sequence complementary to a DNA fragment comprising cytosine sites to be tested in the DNA sample, wherein all the cytosine sites are substituted with thymines.

7. A kit for detecting methylation at cytosine sites in CpG islands in a DNA sample to be tested, which comprises:
(a) the microarray plate of claim 6, and
(b) reagents for bisulfite-conversion and/or DNA labeling reagents.

8. A kit for detecting methylation at cytosine sites in CpG islands in a DNA sample to be tested, which comprises:
(a) an oligonucleotide comprising a sequence complementary to a DNA fragment comprising cytosine sites to be tested in the DNA sample, wherein cytosine sites other than the cytosine sites to be tested are substituted with thymines; and
(b) an oligonucleotide comprising a sequence complementary to a DNA fragment comprising cytosine sites to be tested in the DNA sample, wherein all the cytosine sites are substituted with thymines.
